(19) United States
(12) Patent Application Publication
(10) Pub. No.: US 2016/0113650 A1
(43) Pub. Date: Apr. 28, 2016

(54) Physiologic dresssing for closing wounds

(71) Applicants: Raymond E. Lord, North Adams, MA (US); John M. Lord, North Adams, MA (US)

(72) Inventors: Raymond E. Lord, North Adams, MA (US); John M. Lord, North Adams, MA (US)

(21) Appl. No.: 14/121,827

(22) Filed: Oct. 22, 2014

(51) Int. Cl. A61B 17/08

(52) U.S. Cl. CPC A61B 17/08; A61B 2017/081

(57) ABSTRACT

Medical tape containing hooks or rings is applied to both sides of a wound and the application of medical lace or elastic bands having a physiologic pull are then applied for superior closure.

### BACKGROUND

This invention relates to the field of surgical dressing for closing wounds. More specifically, the invention comprises the use of medical lace or elastic bands creating a physiologic pull for closing wounds replacing conventional stitches requiring sutures.

Another component of the invention is the use of a medical and biologically acceptable tape containing hooks or rings to be placed on both sides of the wound.

Furthermore, the medical lace and elastic bands applied to the wound area accomplish closure and will have a pull matched to the muscle pull in any given anatomical area.

Lastly, the end result is superior closure with the tissue being held in close approximation despite inflammation, muscle contraction and overall healing while no new trauma is introduced in the procedure.

### SUMMARY OF THE INVENTION

It is the object of this invention to provide a quick and easy method for closing wounds while eliminating current suturing issues namely:

A) with inflammation there is some tearing.

B) new trauma is inflicted with suture and staple placement.

C) with muscle contraction of opposing parts there is tearing.

D) with some healing, sutures loosen and are no longer approximating.

### BRIEF DESCRIPTION OF THE DRAWING

FIG. 1, FIG. 5 and FIG. 9 shows in expanded view a closed wound utilizing medical tape containing hooks and elastic bands.

FIG. 2, FIG. 6 and FIG. 10 shows in expanded view a closed wound utilizing medical tape containing rings and lace.

FIG. 3 shows in unassembled view an open wound demonstrating the application using single hooks and elastic band.

FIG. 4 shows in unassembled view an open wound demonstrating the application using single rings and lace.

FIG. 7 shows in unassembled view an open wound demonstrating the application using multiple hooks and elastic band.

FIG. 8 shows in unassembled view an open wound demonstrating the application using multiple rings and lace.

FIG. 9 shows in expanded view a closed irregular wound utilizing medical tape containing hooks and elastic bands.

FIG. 10 shows in expanded view a closed irregular wound utilizing medical tape containing rings and medical lace.

### DESCRIPTION OF THE PREFERRED EMBODIMENTS

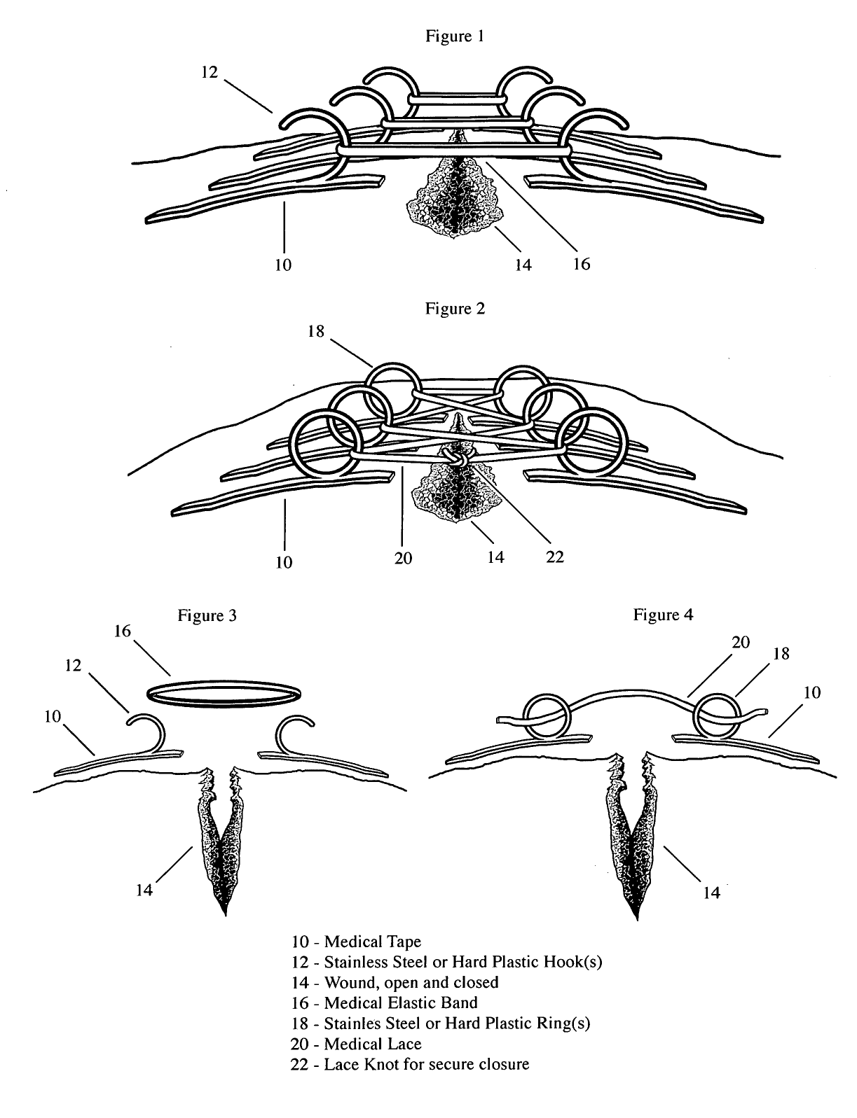

The assembly of the drawing FIG. 1 and FIG. 5 includes medical tape 10 applied to both sides containing hooks 12 to the wound 14 thus applying elastic bands 16 for full closure.

The assembly of the drawing FIG. 2 and FIG. 6 includes medical tape 10 applied to both sides containing rings 18 to the wound 14 thus applying lace 20 tied on one end 22 for full closure.

The drawing FIG. 3 displays a single hook 12 attached to tape 10 on both sides of wound 14 using an elastic band 16 demonstrating technique to be applied.

The drawing FIG. 4 displays a single ring 18 attached to tape 10 on both sides of wound 14 using lace 20 demonstrating technique to be applied.

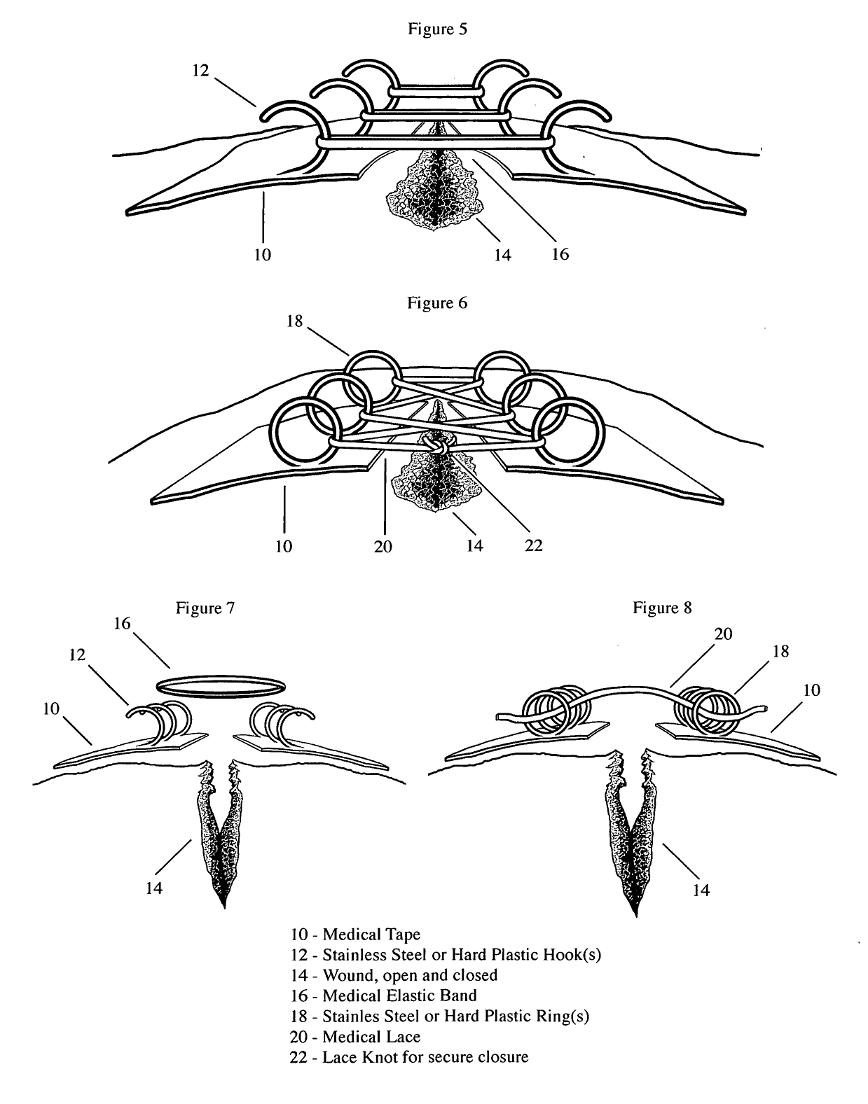

The drawing FIG. 7 displays multiple hooks 12 attached to medical tape 10 on both sides of wound 14 using an elastic band demonstrating technique to be applied.

The drawing FIG. 8 displays multiple rings 18 attached to medical tape 10 on both sides of wound 14 using lace 20 demonstrating technique to be applied.

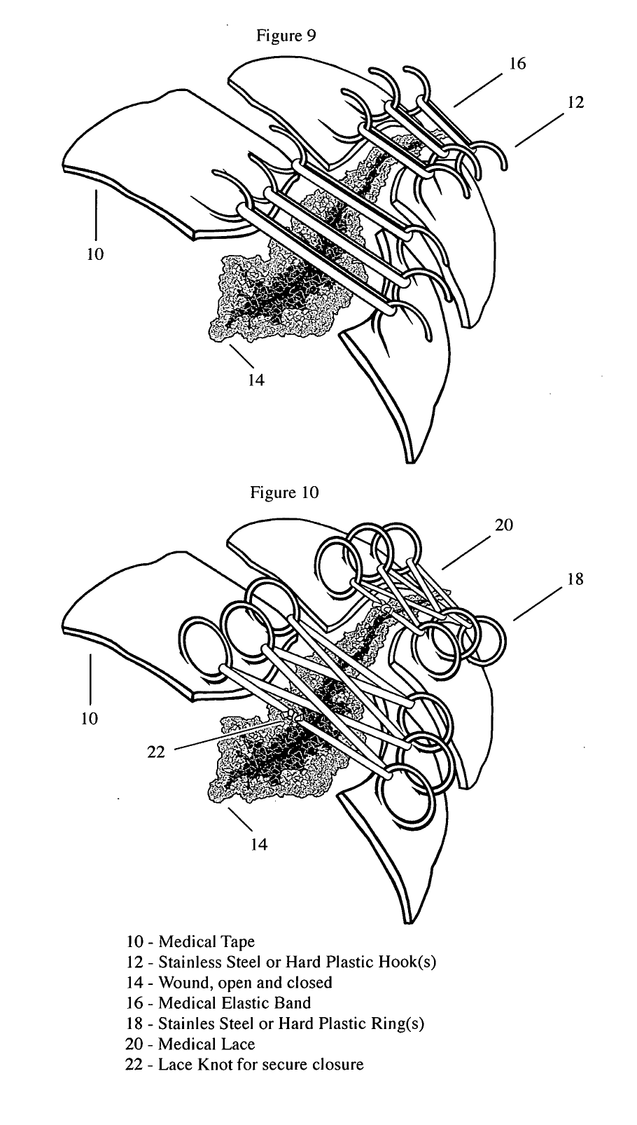

The assembly of drawing FIG. 9 includes tape 10 applied to both sides containing hooks 12 to the irregular wound 14 thus applying elastic bands 16 for full closure.

The assembly of drawing FIG. 10 includes medical tape 10 applied to both sides containing rings 18 to the irregular wound 14 thus applying lace 20 tied on one end 22 for full closure.

I claim:

1. Easy application and removal.

2. No new tissue damage created during the application or healing period.

3. With the procedure there is constant physiologic pull maintaining constant closure.

4. No damage to adjacent tissue.

5. Normal healing time would be accelerated due to physiologic closing pressure.

6. Best method for post healing aesthetics.
